(19) United States
(12) Patent Application Publication
(10) Pub. No.: US 2004/0213809 A1
(43) Pub. Date: Oct. 28, 2004

(54) Method of treating diseases connected with immunity deficiency

(76) Inventor: Yakov Yevseyevich Brilman, Voorhees, NJ (US)

Correspondence Address: YAKOV BRILMAN, 219 Laurel Rd, apt. 508, Voorhees, NJ, 08043, US

(21) Appl. No.: 10/421,660

(22) Filed: Apr. 23, 2003

(51) Int. Cl. A61K 39/02; C12N 1/20

(52) U.S. Cl. 424/234.1; 435/252.3

(57) ABSTRACT

1. In connection with absence necessary laboratory differential-diagnostic test in genus Escherichia and type species E. Coli they are enclosed alongside with nonpathogenic E. Coli as well as pathogenic (EPE). Such is differential-diagnostic test is discovered—oxidation-reduction test (ORT), or dehydrogenase test (DGT), or test of the author (Brilman's test). Characterizing reduction of the indicator during incubation bacteria, that also as beside Salmonellas, Shigellas and other pathogenic bacteria constant negative in semifluid mannitol and in Kligler's medium beside EPE and constant positive beside nonpathogenic E. Coli, that allows to select EPE in separate pathogenic genus bacteria—in genus Brilmanella

2. Found in sick person with dysfunction of the bowels 22 strains “E. Coli” (0.62%) with constantly negative ORT and not referring to known serovar EPE need for additional special study and probably pertain to variety of the genus Brilmanella

According to existing categorizations bacteria in family Enterobacteriaceae is the genus Escherichia with type species E. Coli which are found with nonpathogenic bacteria E. Coli and pathogenic, differing from first in antigenic structure, constant and typical for each strain. Association of these bacteria in one genus, and type species E. Coli is connected with that taken into account morphological and biochemical characteristics like them

Tests allowing differences in these unconnected bacteria was discovered as a result of laboratory observations on significant laboratory material—this is dehydrogenase test (DGT) or oxidation-reduction test (ORT), or test of the author.

In the base bacteriological researches conducted in bacteriological laboratories lies determination ferments hydrolases catalysis hydrolysis of the organic join on more simply, causing hydrolysis disinteration carbohydrate. Herewith, it is not taken into account group oxidized-reconstruction ferments, being the basis of bacteriological breathing and fermentations. In this group enter aerobic and anaerobic dehydrogenases. In the last case role hydrogen acceptor in process of the oxidation execute alongside with other material different paints, which at reaction of the join with hydrogen reduction and are decolorized. Accound of the reduction of the applicable indicator lies in the basis of proposed additional test, which has such diagnostic importance either as determination other ferment, taken into accound in laboratory practical (cytochrome oxidate, dehydrocarboxydase, urease, etc).

Essence of the test is concluded in the following: under incubation bacteria on semifluid ambience Giss and Kligler's medium, beside nonpathogenic Escherichia exists typical biochemical processes, under which the indicator what fixite carbohydrase fermentation the carbohydrate by change of its colouration, restores in process of the incubations initial colour in aerobic layer of these ambiences first, but then and in anaerobic (over 48-72 h. of the incubation).

Oxidation-reduction process is possible fix through 20-24 h. growing bacteria on nourishing ambience Giss (semifluid mannitol) on change the colour of the indicator in its surface layer in contrast with remaining portion of the column of the nourishing ambience. The given test is discovered in the study find beside sick children with dysfunction intestine 2886 strains bacteria E. Coli/disregarding enteropathogenic Escherichia (EPE)/and nearly beside all of them (99.38%) dehydrogenase test (DGT) or (ORT) was positive with the exclusion of 22 strains (0.62%) not refferring to known serovars/complete serotyping include somatic (o), capsular (k), flagellar (h) antigenes/enteropathogenic Escherichia (EPE) and this test was negative.

From found 112 strains different serovars enteropathogenic Escherichia (EPE) beside all of them is the dehydrogenase test was constant negative (absence recovering the colouration of the indicator) in column semi-fluid mannitol and in Kligler's medium, that is to say also as beside Salmonellas, Shigellas, Yersinias and other pathogenic bacteria, for which typical is an absence of dehydrogenase fermentation.

Research on pathogenic intestine sticks conducts a usually applicable method and begins with serological diagnosis. However, considering that, that on Petri dish with applicable to this effect of nourishing substances enteropathogenic intestine sticks do not differ from colony conditionally pathogenic E. Coli and also that, that meet enteropathogenic intestine sticks are not diagnosed by available set of diagnostic serosities but beside which genus OR-test is negative, that are significant to their accessories to pathogenic genus of intestine sticks, follows for account of this test sift with under investigations Petri dishes on three sugar agar and semifluid mannitol (from Glass substance), from each type colonies grew on several of them, irrespective of result the serological research. There sift incubation under 37° C. 24-27 (befor 48) hours whereupon take into account the fermenting characteristics and continue work with bacteria beside which OR-test is negative.

With provision for that constant absence dehydrogenase fermentation beside pathogenic bacteria is a typicaltest for them, which can enter along taken into account generic test, differentiating them from nonpathogenic bacteria allows to select enteropathogenic Escherichia Coli (EPE) in a separate pathogenic genus bacteria family Enterobacteriaceae—in genus Brilmanella.

As to discovered beside sick person with disfunction of the bowels 22 strains bacteria (0.62%) with negative OR-test and not reffering to known serovars enteropathogenic “Escherichia Coli” (EPE) and to other pathogenic Enterobacteria, that is such bacterias need for additional study and there possible are a variety of the pathogenic bacteria genus Brilmanella.

What I claim as my invention is:

1. by my account addition of the laboratory test ORT (DGT) in differencial diagnostics bacteria it is discovered that Enteropathogenic E. Coli is unconnected to nonpathogenic E. Coli, which are wrongly united in one genus E. Coli. It is a separate pathogenic genus bacteria—Brilmanella.
